(19) United States
(12) Patent Application Publication
(10) Pub. No.: US 2007/0233157 A1
(43) Pub. Date: Oct. 4, 2007

(54) FLEXIBLE NEEDLE GUIDE

(76) Inventors: Joseph L. Mark, Indianapolis, IN (US); Zachary R. Nicoson, Indianapolis, IN (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 11/693,534

(22) Filed: Mar. 29, 2007

Related U.S. Application Data
(60) Provisional application No. 60/786,859, filed on Mar. 29, 2006.

(51) Int. Cl. A61B 19/00

(52) U.S. Cl. 606/130

(57) ABSTRACT

An embodiment of a flexible needle guide comprises a support member and a body portion. The body portion is defined by a distal end and a proximal end, wherein the proximal end is connected to the support member. The body portion further includes at least one receiving channel extending therethrough, wherein the receiving channel includes an axis extending therethrough. The body portion is at least partially constructed from a flexible material such that instruments inserted through the receiving channel may be selectively oriented at an angle with respect to the axis extending through the receiving channel.

### CROSS-REFERENCE TO RELATED APPLICATIONS

This application claims priority to U.S. provisional patent application Ser. No. 60/786,859 filed on Mar. 29, 2006, which application is incorporated herein by reference in its entirety.

### BACKGROUND OF THE INVENTION

1. Field of the Invention

The present invention relates generally to needle guides used in biopsy systems and more particularly to a needle guide suitable for guiding a biopsy needle into a patient.

2. Description of the Related Art

During certain biopsy procedures, a needle guide may be used through which a portion of biopsy needle passes. The needle guide serves to minimize non-axial movement or deflection of the biopsy needle during a biopsy to ensure that the biopsy needle is inserted into the correct location of a patient's tissue adjacent a target lesion.

In breast biopsy procedures, such as MRI biopsy procedures, needle guides are often used in connection with a compression plate. One such example of a compression plate 10 is shown in FIGS. 1 and 2. As may be seen, compression plate 10 includes a plurality of grid-like openings 12. Prior to the start of a breast biopsy procedure, the compression plate 10 is pressed against the breast, thereby compressing it, and a needle guide 14 is inserted into one of the openings 12 where the lesion to be biopsied may be found. Known needle guides include a number of apertures that are each sized to permit a portion of a biopsy needle 16 therethrough. The multiple apertures allow for multiple insertion points within each grid opening. However, known needle guides are constructed of stiff unyielding rigid material such that the biopsy needle is limited to an orientation that is at a 90° angle to the compression plate, at all times.

Because the biopsy needle is restricted to a 90° angle orientation to the compression plate, access to breast lesions in some patients is limited. For example, thin-breasted patients, hard to reach chest-wall lesions or close-to-skin target sites may require that the biopsy needle be angled to accomplish biopsy of a lesion, which is not possible with rigid needle guides.

### SUMMARY

An embodiment of a flexible needle guide comprises a support member and a body portion. The body portion is defined by a distal end and a proximal end, wherein the proximal end is connected to the support member. The body portion further includes at least one receiving channel extending therethrough, wherein the receiving channel includes an axis extending therethrough. The body portion is at least partially constructed from a flexible material such that instruments inserted through the receiving channel may be selectively oriented at an angle with respect to the axis extending through the receiving channel.

### BRIEF DESCRIPTION OF THE DRAWINGS

FIG. 1 is a perspective view of a compression plate used in breast biopsy procedures;

FIG. 2 is a perspective view of a compression plate with a needle guide inserted therethrough.

FIG. 3 is a perspective view of an embodiment of a flexible needle guide.

FIG. 4 is an end view of the flexible needle guide of FIG. 3.

FIG. 5 is a side elevational view of the flexible needle guide of FIG. 3.

FIG. 5A is a cross-sectional view of the flexible needle guide taken along lines A-A of FIG. 5,

FIG. 6 is a perspective view of an embodiment of a support member and support flange.

FIG. 6A is a perspective view of an embodiment of a flexible needle guide that incorporates the support member of FIG. 6.

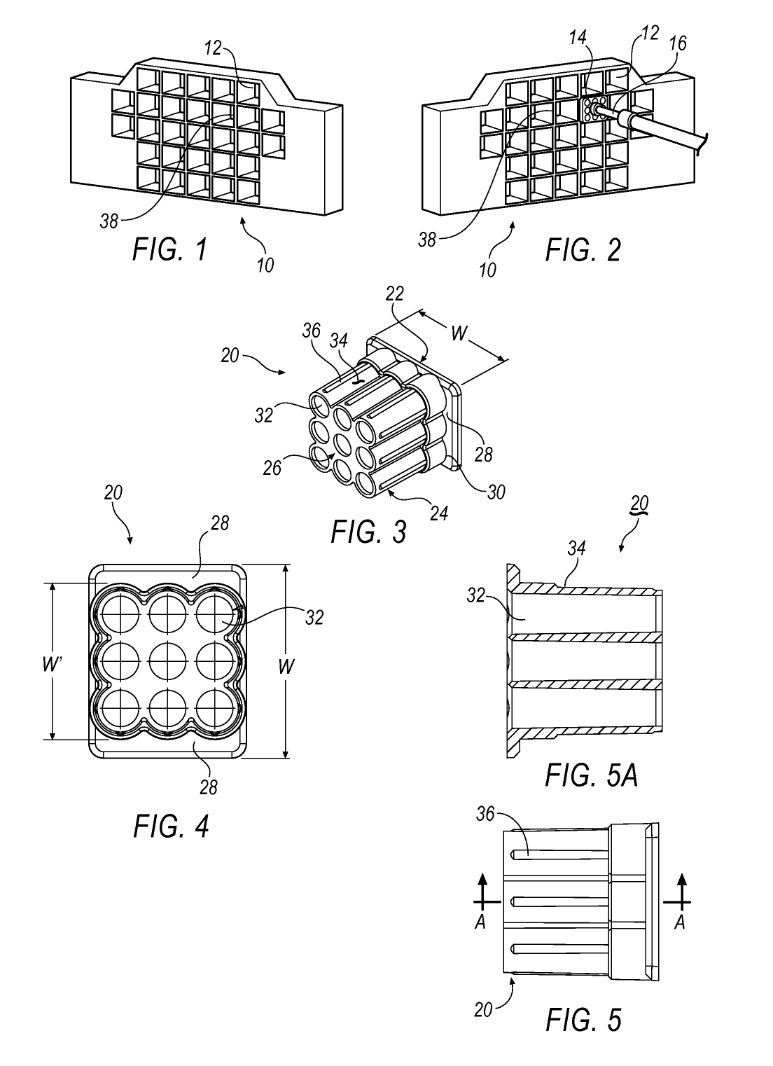

### DETAILED DESCRIPTION

For the purposes of promoting an understanding of the principles of the invention, reference will now be made to the embodiments illustrated in the drawings and specific language will be used to describe the same. It will nevertheless be understood that no limitation of the scope of the invention is thereby intended. The invention includes any alterations and further modifications in the illustrated devices and described methods and further applications of the principles of the invention that would normally occur to one skilled in the art to which the invention relates.

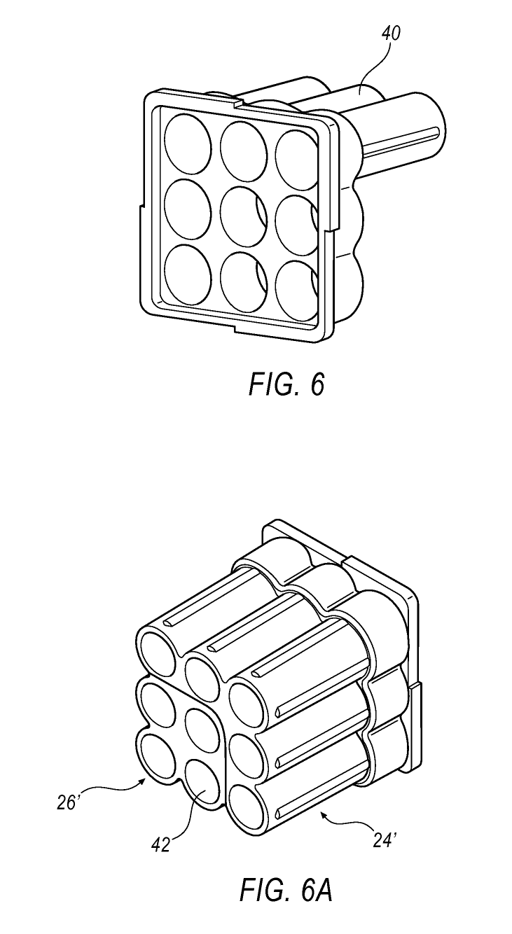

Referring to FIGS. 3-5A, a flexible needle guide 20 is illustrated. Flexible needle guide 20 includes a support member 22, a body portion 24 and a distal end 26. In one embodiment, support member 22 is sized so as to have an overall width w that is at least slightly longer than a width w′ of body portion 24 (see FIG. 4) so as to define mounting shoulders 28 on either side of body portion 24, as will be explained in greater detail below. While the embodiment of FIG. 3 illustrates the mounting shoulders 28 as extending the entire length of body portion, it is understood that mounting shoulders 28 need not extend the entire length of body portion (see, e.g., FIG. 6).

Body portion 24 is defined by distal end 26 and a proximal end 30 that is connected to support member 22. In one embodiment, proximal end 30 is integrally formed with support member 22. Further, support member 22 may also be provided with a support flange 23 that extends around proximal end 30 of body portion for added support. Support flange 23 may also be integrally formed with support member 22 or body portion 24, or both.

Body portion 24 includes at least one receiving channel 32 formed therethrough. In one embodiment, body portion 24 includes a plurality of channels 32 arranged to form a honeycomb configuration. It is understood, however, that body portion 24 may include any number of channels 32.

Disposed on an outside surface 34 of body portion 24 are one or more retaining members 36. In one embodiment, retaining members 36 are ridges that extend away from the outside surface 34 of body portion 24. Retaining members 36 may be integrally formed with body portion 24. The purpose of retaining members 36 will be explained in greater detail below.

In operation, needle guide 20 is inserted into one of the openings 12 formed in compression plate 10, as shown in FIG. 2, with distal end 26 extending through opening 12; Support member 22 is oriented such that mounting shoulders 28 extend across walls 38 of compression plate 10 that define adjacent grid openings 12. Thus, mounting shoulders 28 ensure that needle guide 20 does not extend completely through opening 12.

The retaining members 36 frictionally engage the walls 38 that define grid openings 12. Retaining members 36 serve to hold needle guide 20 in place during procedures.

While known needle guides have been constructed of a rigid material, the prior art needle guides restricted a physician's placement of a biopsy needle or other surgical instrument, because the biopsy needle was forced to remain at a 90° angle to the compression plate 10 at all times due to the rigidity of the prior art needle guides.

In contrast, needle guide 20 is constructed, at least in part, of a flexible material, such as a rubber material, having a lower durometer than known rigid needle guides. One suitable material is a copolymer of ethylene and octene-1, sold under the tradename ENGAGE® BY DuPont Dow Elastomers L.L.C.

In one embodiment, support member 22 and support flange 23 are constructed of a relatively high durometer material, while body portion 24 is constructed of a lower durometer material. In one embodiment, the high durometer material is approximately 80-100 durometer. In one embodiment, the lower durometer material is approximately 35-55 durometer. By constructing the needle guide 20 at least partially from a flexible material, needles and other instruments inserted into one of the receiving channels 32 can be manipulated to different angles. More specifically, the distal end 26 of the body portion 24 is very flexible and can be bent so as to allow for up a 30° angle deviation of the biopsy device while the device is positioned within one of the channels 32. However, while allowing flexibility, needle guide 20 is still semi-rigid enough to provide a traditional 90° angle access when desired.

Use of the flexible need guide allows for improved selective needle position to provide treatment options for patients that otherwise may not be able to receive minimally invasive biopsy and/or therapy procedures, such as thin-breasted patients, hard to reach chest-wall or close to skin target sites. Thus, insertion location, depth, and needle angle may be more accurately accomplished.

In another embodiment, shown in FIGS. 6 and 6A, body portion 24′ may include a substantially rigid frame 40 (shown in FIG. 6) constructed of relatively high durometer material that forms part of the body portion 24.′ The remainder of the body portion 24′ includes a flexible material 42 of a lower durometer material than frame 40 overmolded thereto. In this embodiment, increased rigidity ensures retention of needle guide 20 within opening 12 of the compression plate 10, but flexibility is still provided in certain portions of the needle guide, especially at the distal end 26′ to permit selective placement of a biopsy needle or other medical instrument.

Although certain preferred embodiments of the present invention have been described, the invention is not limited to the illustrations described and shown herein, which are deemed to be merely illustrative of the best modes of carrying out the invention. A person of ordinary skill in the art will realize that certain modifications and variations will come within the teachings of this invention and that such variations and modifications are within the spirit and the scope as defined by the claims.

What is claimed is:

1. A flexible needle guide, comprising:
a support member; and
a body portion defined by a distal end and a proximal end, wherein the proximal end is connected to the support member;
wherein the body portion further includes at least one receiving channel extending therethrough, wherein the receiving channel includes an axis extending therethrough; and
wherein the body portion is at least partially constructed from a flexible material such that instruments inserted though the receiving channel may be selectively oriented at an angle with respect to the axis extending through the receiving channel.

2. The flexible needle guide of claim 1, wherein the support member further defines one or more mounting shoulders thereon.

3. The flexible needle guide of claim 2, wherein the support member defines two mounting shoulders thereon, wherein one mounting shoulder is positioned on a first side of body portion and a second mounting shoulder is positioned on an opposite side of body portion.

4. The flexible needle guide of claim 3, wherein the mounting shoulders extend the entire length of the body portion.

5. The flexible needle guide of claim 1, wherein the body portion is constructed entirely of the flexible material.

6. The flexible needle guide of claim 1, further including a support flange connected to the support member, wherein said support flange extends around the proximal end of the body portion.

7. The flexible needle guide of claim 6, wherein the support flange is integrally formed with the support member.

8. The flexible needle guide of claim 6, wherein the support flange and the support member are construction of a substantially rigid material having a first durometer value.

9. The flexible needle guide of claim 8, wherein the remainder of the body portion is construction of a second material having a second durometer value that is lower than the first durometer value.

10. The flexible needle guide of claim 1, wherein the body portion is at least partially constructed of a flexible copolymer.

11. The flexible needle guide of claim 1, wherein the body portion further includes a substantially rigid frame that extends the length of the body portion and wherein the flexible material is overmolded to the rigid frame to form the body portion.

12. The flexible needle guide of claim 1, further including one or more retaining ridges positioned on an outside surface of the body portion.

13. A flexible needle guide, comprising:
a support member having one or more mounting shoulders thereon;
a support flange; and
a body portion defined by a distal end and a proximal end, wherein the proximal end is connected to the support member and wherein the support flange extends at least partially around the body portion;
wherein the body portion further includes at least one receiving channel extending therethrough, wherein the receiving channel includes an axis extending therethrough; and
wherein the body portion is at least partially constructed from a flexible material such that instruments inserted though the receiving channel may be selectively oriented at an angle with respect to the axis extending through the receiving channel.

14. The flexible needle guide of claim 13, wherein the body portion is constructed entirely of the flexible material.

15. The flexible needle guide of claim 13, wherein the support flange and the support member are construction of a substantially rigid material having a first durometer value.

16. The flexible needle guide of claim 15, wherein the remainder of the body portion is construction of a second material having a second durometer value that is lower than the first durometer value.

17. The flexible needle guide of claim 13, wherein the body portion further includes a substantially rigid frame that extends the length of the body portion and wherein the flexible material is overmolded to the rigid frame to form the body portion.

18. The flexible needle guide of claim 13, further including one or more retaining ridges positioned on an outside surface of the body portion.
